(19) United States
(12) Patent Application Publication
(10) Pub. No.: US 2026/0248856 A1
(43) Pub. Date: Aug. 27, 2026

(54) SOMATIC STEM CELLS AND USE THEREOF FOR IMMUNOMODULATION

(71) Applicant: StemBios Technologies, Inc., Covina, CA (US)

(72) Inventors: Yun Yen, Arcadia, CA (US); Shu-Ching Hsu, Taipei City (TW)

(21) Appl. No.: 19/546,927

(22) Filed: Feb. 23, 2026

Related U.S. Application Data
(60) Provisional application No. 63/762,014, filed on Feb. 23, 2025.

(51) Int. Cl. A61K 35/28; A61K 40/10; A61K 40/42; A61P 37/06; C07K 14/705; C12N 5/0775

(52) U.S. Cl. CPC A61K 35/28; A61K 40/10; A61K 40/421; A61P 37/06; C07K 14/70532; C12N 5/0663

(57) ABSTRACT

The disclosure is drawn to an isolated somatic stem cell population including somatic stem cells that are PD-L1+ and methods of using the isolated somatic stem cell population to modulate an immune response.

### RELATED APPLICATION

This application claims the benefit of U.S. Provisional Application No. 63/762,014, filed on Feb. 23, 2025. The entire teaching of the above application is incorporated herein by reference.

### BACKGROUND

Stem cells are known as totipotent or pluripotent precursor cells capable of generating a variety of mature human cell lineages. Among various sources of stem cells, somatic stem cells have been proposed for various therapeutic applications.

It is widely known that the immune system consists of multicellular interactions that culminate in the inhibition, clearance, and induction of memory to agents that are a threat to the normal functioning of the host. However, many pathologies manifest clinically through unwanted or excessive immune responses within a host, e.g., transplant rejection, inflammatory and autoimmune disorders. Immunosuppressive therapies have been developed to treat the symptoms, but not the underlying cause of pathologies characterized by excessive immune responses. These therapies are effective at down-modulating immune function and, as such, carry the potential for severe adverse events, including cancer and opportunistic infection, as well as side effects such as cataracts, hyperglycemia, bruising, and nephrotoxicity from agents such as prednisone, cyclosporine, and tacrolimus.

There is a need to uncover and develop stem cells with superior immunomodulation ability to increase therapeutic efficacy.

### SUMMARY

In one aspect, provided herein is an isolated somatic stem cell population, comprising somatic stem cells that are about 6 μm or less in size and are PD-L1+.

In some embodiments, at least about 50% (e.g., at least about 50% to 99%) of the cells in the somatic stem cell population are PD-L1+. In some embodiments, at least 80% of the cells in the isolated somatic stem cell population are about 6 μm or less in size.

In another embodiment, the isolated somatic stem cell population further comprises somatic stem cells that are about 6 μm or less in size and are Lgr5+. In some embodiments, at least about 50% of the Lgr5+ cells are PD-L1+.

In yet another embodiment, at least about 80% of the cells in the isolated somatic stem cell population are PD-L1+.

In some embodiments, the isolated somatic stem cell population further comprises somatic stem cells that are CD61+, HLA-ABC+, CD69+, and/or CD127+.

In an embodiment, the isolated somatic stem cell population is prepared using a process including: incubating a peripheral blood or bone marrow sample with EDTA or heparin in a container until the sample is separated into an upper layer and a lower layer; collecting the upper layer; and isolating from the upper layer a population of somatic stem cells containing somatic stem cells that are about 6.0 μm or less in size and PD-L1+.

In another aspect, a composition comprising the isolated somatic stem cell population of the present disclosure is provided.

In yet another aspect, described herein is a method of modulating an immune response, comprising contacting immune cells with an effective amount of the isolated somatic stem cell population of this disclosure. In one embodiment, the immune cells are T cells, and the immune response is suppressed.

In some embodiments, the contacting step in the method is carried out in vitro.

In other embodiments, the contact step in the method includes administering an effective amount of the isolated somatic stem cell population to a subject in need thereof. The isolated somatic stem cell population may be allogeneic or autologous.

In some embodiments, the subject being administered with an effective amount of the isolated somatic stem cell population has or is at risk of developing an alloresponse or autoimmune response. In some embodiments, the subject has or is at risk of developing an autoimmune disorder (e.g., inflammatory bowel disease, graft-versus-host disease, rheumatoid arthritis, multiple sclerosis, systemic lupus erythematosus, scleroderma, diabetes or psoriasis).

### BRIEF DESCRIPTION OF THE DRAWINGS

FIG. 1 includes a set of graphs showing characterization of SB cells. SB cells were isolated from human peripheral blood. The expression of stemness markers (Lgr5 and CD34), as well as several hematological (CD45, CD3, CD19, CD34, and CD61) and immune-property (HLA-ABC, CD44, CD69, CD127, PD-L1, TIM3, LAG3, and CTLA4) markers in SB cells were examined via FACS analysis. The representative (A) and statistical (B) results of the Lgr5+ population after isolation from 6 independent donors are shown. Error bar, S.E.M. n=6, (two-sided unpaired t-test, ***p<0.001). The representative (C) and statistical (D) results of the FACS analysis for hematological and immune-property markers in both Lgr5− and Lgr5+ populations of SB cells are shown. Error bar, S.E.M. n=6, (two-sided unpaired t-test, **p<0.01, ***p<0.001).

FIG. 2 is a set of graphs showing immunogenicity and immunomodulating properties of SB cells. (A) For immunogenicity validation, SB cells were co-cultured with autogenous or allogenic T cells at a 1:1 or 10:1 (SB cells to T cells) ratio for 3 days. To determine cell proliferation activity after co-culturing, [3H]-thymidine (1 μCi) was added to each well on day 2, and the cell cultures were harvested on day 3 to measure the radioactivity (CPM). T cells were activated by anti-CD3 (1 μg/mL) and anti-CD28 (1 μg/mL) antibodies (αCD3/αCD28) for 3 days as positive control. Data (CPM) were calculated as fold of control relative to the counts of the control group (CTR, T cells alone without activation). Error bar, S.E.M. n=6, (two-sided unpaired t-test, ns, not significant, ***p<0.001). (B) For immunomodulating validation, SB cells were co-cultured with T cells as described above but with the activation using αCD3/αCD28. Cells were added with [3H]-thymidine (1 μCi) on day 2 and harvested on day 3 of co-culturing, and the radioactivity of each well was determined (CPM). Data (CPM) were calculated as the percentage (%) of T cells alone group. Error bar, S.E.M. n=6, (two-sided unpaired t-test, *p<0.05, ***p<0.001).

FIG. 3 is a set of graphs showing induction of PD-1 population in activated T cells upon co-culturing with SB cells. Human T cells were activated by anti-CD3 (1 μg/mL) and anti-CD 28 (1 μg/mL) antibodies for 3 days with (A) or without (B) the co-culturing of SB cells. Total cells were harvested after co-culturing and the expression of T cell activation (CD69) and immune checkpoint inhibitors (PD-1 and CTLA4) markers were determined in the T cell population (CD3+ cells) via FACS analysis. The representative (A and B) and statistical results (C and D) of the FACS analysis are shown. Error bar, S.E.M. n=6, (two-sided unpaired t-test to T cell alone group, ns, not significant, *p<0.05, **p<0.01).

FIG. 4 is a set of graphs showing that PD-L1+ cells in the SB cell population harbor immunosuppressive activity. (A) Three cell populations (P1: Lgr5−/PD-L1−; P2: Lgr5+/PD-L1+; and P3: Lgr5−/PD-L1+) were isolated from the SB cell population (Total SB cells) by sorting. (B) The SB cell population and the isolated subpopulations were individually co-cultured with T cells under a 10 to 1 (SB cells to T cells) ratio for 3 days, with T cell activation (αCD3/αCD28). Cells were added with [3H]-thymidine (1 μCi) on day 2 and harvested on day 3 of co-culturing, and the radioactivity of each well was determined (CPM). Data (CPM) was calculated as the percentage (%) of suppression compared to the control group (T cells alone). Error bar, S.E.M. n=3, (two-sided unpaired t-test, *p<0.05, **p<0.01, ***p<0.001).

### DETAILED DESCRIPTION

This disclosure describes the unexpected discovery and preparation of an isolated population of somatic stem cells characterized by positive expression of PD-L1. It was also unexpectedly discovered that the population of somatic stem cells exhibits immunomodulation properties (e.g., immunosuppression) and can be used for the immunomodulation and treatment of immune disorders. The data show that the immunomodulation properties of the stem cell population are associated with positive expression of PD-L1.

### Definitions

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon.

The terms “decrease”, “reduced”, “reduction”, “suppress”, and “inhibit” are all used herein generally to mean a decrease by a statistically significant amount. In some embodiments, the terms mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100%, or any decrease between 10-100% as compared to a reference level.

The terms “increased”, “increase”, “enhance”, and “activate” are all used herein generally to mean an increase by a statically significant amount. In some embodiments, the terms mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. As used herein, the term “treatment” includes prophylaxis. The individual is usually ill or injured, or at increased risk of becoming ill relative to an average member of the population and in need of such attention, care, or management. Those in need of treatment include but are not limited to those already diagnosed with an immune disorder or having overactive immune responses, such an inflammatory or autoimmune disorder or showing one or more symptoms of the disorder, as well as those likely to develop the disorder due to genetic susceptibility or other risk factors. One of skill in the art realizes that a treatment may improve a disease condition but may not be a complete cure for the disease.

As used herein, an “effective amount” of a drug, agent, compound, pharmaceutical composition or a cell population is an amount sufficient to produce one or more desired or beneficial results (e.g., clinical results, non-clinical results, phenotypes, or changes in biological functions or processes), such as a reduction in at least one symptom of a disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of the disease, delay or slowing of disease progression, amelioration or palliation of the disease state, prolonging survival, and remission (whether partial or total), whether detectable or undetectable.

As interchangeably used herein, the terms “individual,” “subject,” and “patient,” refer to an animal, such as a mammal. Examples of a subject include human, non-human primates, rodents, guinea pigs, rabbits, sheep, pigs, goats, cows, horses, dogs, and cats.

As used herein, the term “contacting” is intended to include incubating a cell and an agent or another cell together in vitro (e.g., adding the agent or other cells to cells in culture). In some embodiments, the term “contacting” is not intended to include the in vivo exposure of cells to the compounds or cells that may occur naturally in a subject (i.e., exposure that may occur as a result of a natural physiological process). In some embodiments, the term is intended to include the in vivo exposure of cells to the compounds or other cells.

As used herein, “stem cells” are cells that possess self-renewal ability and multiple differentiation ability when exposed to specific environmental conditions. Self-renewal means that during cell division, at least one of the two daughter cells will be a stem cell.

The term “substantially pure” or “substantially homogenous”, when used interchangeably in reference to a cell population, means that the specified cells (e.g., cells positively or negatively expressing a particular cell marker) constitute a majority of the cells in the preparation (e.g., at least about 50% to 99%, at least 50%, at least 60%, at least 65%, at least 70%, at least 75%, at least 80%, at least 85%, at least 90%, at least 95%, or at least 99%). The term “substantially free”, when used in reference to a cell population, means that the specified cells (e.g., cells expressing a particular cell marker) constitute less than about 30%, 25%, 20%, 15%, 10%, 9%, 8%, 7%, 6%, 5%, 4%, 3%, 2%, 1%, or even 0% of the cells in the cell population.

As used herein, “autologous” means that a biological (e.g., a cell population or tissue) is derived from an individual's own body.

As used herein, “allogeneic” refers to tissues or cells from another body that in a natural setting are immunologically incompatible or capable of being immunologically incompatible, although from one or more individuals of the same species.

The terms “proliferation” and “expansion” as used interchangeably herein with reference to cells, refer to an increase in cell number.

The term “differentiation” refers to a developmental process whereby cells become specialized for a particular function, for example, where cells acquire one or more morphological characteristics and/or functions different from that of the initial cell type. The term includes both lineage commitment and terminal differentiation processes.

As used herein, “cell surface marker” means a protein expressed on the surface of a cell which is detectable via specific antibodies.

As used herein, “positive for expression” means that a marker of interest, whether intracellular or extracellular, is detectable in or on a cell using any method, including but not limited to flow cytometry. The terms “positive for expression”, “positively expressing”, “expressing”, and “+” are used interchangeably herein. In some embodiments, the term as applied to a population of cells can mean that the cell population is substantially pure with respect to cells expressing the marker of interest. In some embodiments, the term as applied to a population of cells can mean that expression of the marker is detectable in the population.

As used herein, “negative for expression” means that the marker of interest, whether intracellular or extracellular, is not detectable in or on a cell using any method, including but not limited to flow cytometry. The terms “negative for expression”, “negative expressing”, “not expressing”, and “−” are used interchangeably herein. In some embodiments, the term as applied to a population of cells can mean that expression of the marker is detectable in the population, but the cell population is substantially free of cells expressing the marker.

As used herein, the term “isolated” used in reference to a single cell or cell population means that the cell or cell population is substantially free of other cell types or cellular material with which it naturally occurs in its or their origin. An isolated cell or cell population can have markedly different characteristics in structure, biological or pharmacological function and/or other properties as compared to naturally existing cell or cell population.

As used herein, the term “cell population” or “population of cells”, depending on the context, includes a population of cells that is substantially homogenous. In some embodiments, the term may refer to a heterogeneous cell population.

As used herein, “immunomodulation” and “immunomodulatory” mean causing, or having the capacity to cause, a detectable change in an immune response.

As used herein, “immunosuppression” and “immunosuppressive” mean causing, or having the capacity to cause, a detectable reduction in an immune response, and the ability to cause a detectable suppression of an immune response.

The singular forms “a,” “an” and “the” include plural referents unless the context clearly dictates otherwise. Thus, unless otherwise required by context, singular terms shall include the plural and plural terms shall include the singular.

### Isolated Somatic Stem Cell Population

Described herein is an isolated somatic stem cell population including somatic stem cells that are about 6 μm or less in size (e.g., diameter) and express PD-L1. In some embodiments, the isolated somatic stem cell population further includes Lgr5+ stem cells or Lgr5+/PD-L1+ stem cells. In some embodiments, the isolated stem cell population also contains stem cells that express one or more of CD61, HLA-ABC, CD69, and CD127. In one embodiment, the isolated stem cell population are characterized by the negative expression of CD45, CD3, CD19, CD34, CD44, TIM3, LAG3, and/or CTLA4, e.g., being substantially free of cells expressing one or more of these markers.

SB cells or the SB cell population, as used herein, refers to an isolated population of somatic stem cells that are about 6 μm or less (e.g., 0.3-6.0 micrometer) in size prepared using the process described herein and previously. See, e.g., US2012/0034194 and US2014/0161774. The isolated SB cell population has markedly different characteristics from naturally existing cells.

It was unexpectedly discovered that an isolated subpopulation of somatic stem cells characterized by positive expression of PD-L1 or co-expression of PD-L1 and Lgr5 can be prepared from SB cells. The subpopulation is functionally and structurally different from the SB cell population and other subpopulations.

Antibodies can be used to recognize surface molecules differentially expressed on target cells. The cell surface marker means a protein expressed on the surface of a cell, which is detectable via specific antibodies. Selective methods known in the art and described herein can be used to characterize the isolated somatic stem cell population. A sample containing cells can be reacted with monoclonal/polyclonal antibodies. Subpopulations of cells expressing cell surface antigens can be labeled by fluorescence materials such quantum dots, or labeled by secondary antibodies or be either positively or negatively selected by immunomagnetic bead elucidation, complement mediated lysis, agglutination methods, commercial antibody array kits or fluorescence activated cell sorting (FACS). The cells can also be subjected to functional analysis. Cell markers as well as surface markers that are useful in the disclosure include, but are not limited to, PD-L1 and Lgr5. Exemplary markers are illustrated as follows.

Lgr5 is a leucine-rich repeat-containing receptor (LGR) and member of the G protein-coupled, 7-transmembrane receptor (GPCR) superfamily. Lgr5 is a receptor for R-spondins and is involved in the canonical Wnt signaling pathway. This protein plays a role in the formation and maintenance of adult intestinal stem cells during postembryonic development.

CD3 is a protein complex and T cell co-receptor involved in activating both the cytotoxic T cell (CD8⁺ naive T cells) and T helper cells (CD4⁺ naive T cells).

The PD-L1 marker, or Programmed Death-Ligand 1, is a protein known to significantly influences the immune system's capacity to combat cancer. It is found on the surface of tumor cells and can suppress T-cell activation, enabling cancer cells to escape detection by the immune system. Assessing PD-L1 levels in tumor tissue is essential for identifying patients who may respond positively to immunotherapy, especially treatments that focus on the PD-1/PD-L1 pathway, such as immune checkpoint inhibitors.

B-lymphocyte antigen CD19, commonly referred to as the CD19 molecule (Cluster of Differentiation 19), B-Lymphocyte Surface Antigen B4, T-Cell Surface Antigen Leu-12, and CVID3, is a transmembrane protein encoded by the CD19 gene in humans.

CD34 is a transmembrane phosphoglycoprotein that is encoded by the CD34 gene found in humans, mice, rats, and various other species.

Integrin beta-3 (β3), also known as CD61, is a protein encoded by the ITGB3 gene in humans. CD61 is a cluster of differentiation present on platelets (thrombocytes).

The human leukocyte antigen (HLA) system consists of a group of genes located on chromosome 6 that encode proteins on the surface of cells, playing a crucial role in the regulation of the immune system. This system is essentially the human equivalent of the major histocompatibility complex (MHC) found in various animal species.

CD69 is a membrane-bound, type II C-lectin receptor. It is a classical early marker of lymphocyte activation due to its rapid appearance on the surface of the plasma membrane after stimulation. CD69 is expressed by several subsets of tissue resident immune cells, including resident memory T (TRM) cells and gamma delta (γδ) T cells, and is therefore considered a marker of tissue retention.

CD45, also known as protein tyrosine phosphatase, receptor type, C and PTPRC, is an enzyme that, in humans, is encoded by the PTPRC gene. CD45RA is located on naive T cells and CD45RO is located on memory T cells. CD45 is also highly glycosylated. CD45RA is located on naive T cells. Naive T lymphocytes express large CD45 isoforms and are usually positive for CD45RA. CD45RO facilitates T cell activation.

CTLA4 (cytotoxic T-lymphocyte-associated protein 4), also known as CD152 (cluster of differentiation 152), is a protein receptor that functions as an immune checkpoint and downregulates immune responses.

CD127, also known as interleukin-7 receptor, is a protein found on the surface of cells, including naive and memory T cells and many others. CD127 has been shown to play a critical role in the development of lymphocytes.

CD44 is a cell-surface glycoprotein involved in cell-cell interactions, cell adhesion and migration. CD44 participates in a wide variety of cellular functions including lymphocyte activation, recirculation and homing, hematopoiesis, and tumor metastasis.

Lymphocyte-activation gene 3, commonly referred to as LAG-3, is a protein encoded by the LAG3 gene in humans.

Hepatitis A virus cellular receptor 2 (HAVCR2), commonly referred to as T-cell immunoglobulin and mucin-domain containing-3 (TIM-3), is a protein encoded by the HAVCR2 (TIM-3) gene in humans.

In some embodiments, the lack of expression of a cell surface marker defines the isolated somatic stem cell population of the disclosure.

In some embodiments, at least about 50% to 99% (e.g., at least 50%, at least 60%, at least 65%, at least 70%, at least 75%, at least 80%, at least 85%, at least 90%, at least 95%, at least 99%, about 55%, about 60%, about 70%, about 75%, about 80%, about 85%, about 90%, about 91%, about 92%, about 93%, about 94%, about 95%, about 96%, about 97%, about 98%, or about 99%) of the cells within the isolated somatic stem cell population of the disclosure positively or negatively express a cell marker of interest (e.g., PD-L1, Lgr5, CD45, CD3, CD19, CD34, CD61, HLA-ABC, CD44, CD69, CD127, TIM3, LAG3, or CTLA4); in yet other embodiments, at least about 90%, at least about 95%, about 96%, about 97%, about 98%, about 99%, or even about 100% of the cells within the isolated somatic stem cell population of the disclosure positively or negatively express a cell marker of interest (e.g., PD-L1, Lgr5, CD45, CD3, CD19, CD34, CD61, HLA-ABC, CD44, CD69, CD127, TIM3, LAG3, or CTLA4).

In some embodiments, at least about 50% to 99% (e.g., at least 50%, at least 60%, at least 65%, at least 70%, at least 75%, at least 80%, at least 85%, at least 90%, at least 95%, or at least 99%, about 55%, about 60%, about 70%, about 75%, about 80%, about 85%, about 90%, about 91%, about 92%, about 93%, about 94%, about 95%, about 96%, about 97%, about 98%, or about 99% of the cells within the isolated somatic stem cell population of the disclosure express PD-L1. In some embodiments of a substantially pure somatic stem cell population with respect to positive PD-L1 expression (e.g., at least about 50% to 99% of the cells in the population are PD-L1+), at least about 25% to 99% (e.g., at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, about 55%, about 60%, about 70%, about 75%, about 80%, about 85%, about 90%, about 91%, about 92%, about 93%, about 94%, about 95%, about 96%, about 97%, about 98%, or about 99%) of the cells within the isolated somatic stem cell population of the disclosure express Lgr5+.

In yet another embodiment, at least about 50% to 99% (e.g., at least 50%, at least 60%, at least 65%, at least 70%, at least 75%, at least 80%, at least 85%, at least 90%, at least 95%, or at least 99%, about 55%, about 60%, about 70%, about 75%, about 80%, about 85%, about 90%, about 91%, about 92%, about 93%, about 94%, about 95%, about 96%, about 97%, about 98%, or about 99% of the cells) within the isolated somatic stem cell population of the disclosure express Lgr5. In some embodiments of a substantially pure somatic stem cell population with respect to positive Lgr5 expression (e.g., at least about 50% to 99% of the cells in the population are Lgr5+), at least about 50% (e.g., at least 50%, at least 60%, at least 65%, at least 70%, at least 75%, at least 80%, at least 85%, at least 90%, at least 95%, at least 99%, about 55%, about 60%, about 70%, about 75%, about 80%, about 85%, about 90%, about 91%, about 92%, about 93%, about 94%, about 95%, about 96%, about 97%, about 98%, or about 99%) of the cells within the isolated somatic stem cell population express PD-L1.

In some embodiments, the isolated somatic stem cell population of the disclosure further includes cells that express one of more of CD61+, HLA-ABC+, CD69+, and CD127+. For example, within the isolated somatic stem cell population at least about 50% to 99% (e.g., at least 50%, at least 60%, at least 65%, at least 70%, at least 75%, at least 80%, at least 85%, at least 90%, at least 95%, or at least 99%, about 55%, about 60%, about 70%, about 75%, about 80%, about 85%, about 90%, about 91%, about 92%, about 93%, about 94%, about 95%, about 96%, about 97%, about 98%, or about 99%) express CD61; at least about 50% to 99% (e.g., at least 50%, at least 60%, at least 65%, at least 70%, at least 75%, at least 80%, at least 85%, at least 90%, at least 95%, or at least 99%, about 55%, about 60%, about 70%, about 75%, about 80%, about 85%, about 90%, about 91%, about 92%, about 93%, about 94%, about 95%, about 96%, about 97%, about 98%, or about 99%) express HLA-ABC; at least about 50% to 99% (e.g., at least 50%, at least 60%, at least 65%, at least 70%, at least 75%, at least 80%, at least 85%, at least 90%, at least 95%, or at least 99%, about 55%, about 60%, about 70%, about 75%, about 80%, about 85%, about 90%, about 91%, about 92%, about 93%, about 94%, about 95%, about 96%, about 97%, about 98%, or about 99%) express CD69+; and/or at least about 50% to 99% (e.g., at least 50%, at least 60%, at least 65%, at least 70%, at least 75%, at least 80%, at least 85%, at least 90%, at least 95%, or at least 99%, about 55%, about 60%, about 70%, about 75%, about 80%, about 85%, about 90%, about 91%, about 92%, about 93%, about 94%, about 95%, about 96%, about 97%, about 98%, or about 99%) express CD127.

In one embodiment, the isolated somatic stem cell population of the disclosure is substantially free of cells expressing one or more of CD45, CD3, CD19, CD34, CD44, TIM3, LAG3, and CTLA4. In some embodiments, within the isolated somatic stem cell population, about 15% or less (e.g., less than 15%, less than 10%, less than 9%, less than 8%, less than 7%, less than 6%, less than 5%, less than 4%, less than 3%, less than 2%, or less than 1%) express CD45; about 15% or less (e.g., less than 15%, less than 10%, less than 9%, less than 8%, less than 7%, less than 6%, less than 5%, less than 4%, less than 3%, less than 2%, or less than 1%) express CD3; about 15% or less (e.g., less than 15%, less than 10%, less than 9%, less than 8%, less than 7%, less than 6%, less than 5%, less than 4%, less than 3%, less than 2%, or less than 1%) express CD19; about 15% or less (e.g., less than 15%, less than 10%, less than 9%, less than 8%, less than 7%, less than 6%, less than 5%, less than 4%, less than 3%, less than 2%, or less than 1%) express CD34; about 15% or less (e.g., less than 15%, less than 10%, less than 9%, less than 8%, less than 7%, less than 6%, less than 5%, less than 4%, less than 3%, less than 2%, or less than 1%) express CD44; about 15% or less (e.g., less than 15%, less than 10%, less than 9%, less than 8%, less than 7%, less than 6%, less than 5%, less than 4%, less than 3%, less than 2%, or less than 1%) express TIM3; about 15% or less (e.g., less than 15%, less than 10%, less than 9%, less than 8%, less than 7%, less than 6%, less than 5%, less than 4%, less than 3%, less than 2%, or less than 1%) express LAG3; and/or about 15% or less (e.g., less than 15%, less than 10%, less than 9%, less than 8%, less than 7%, less than 6%, less than 5%, less than 4%, less than 3%, less than 2%, or less than 1%) express CTLA4.

In some embodiments, the isolated somatic stem cell population of the disclosure is characterized by positive expression of PD-L1, Lgr5, CD61+, HLA-ABC+, CD69+, and CD127+, and negative expression of CD45, CD3, CD19, CD34, CD44, TIM3, LAG3, and CTLA4. For example, at least about 50% to 99% (e.g., at least 50%, at least 60%, at least 65%, at least 70%, at least 75%, at least 80%, at least 85%, at least 90%, at least 95%, or at least 99%, about 55%, about 60%, about 70%, about 75%, about 80%, about 85%, about 90%, about 91%, about 92%, about 93%, about 94%, about 95%, about 96%, about 97%, about 98%, or about 99% of the cells within the isolated somatic stem cell population of the disclosure express PD-L1, Lgr5, CD61+, HLA-ABC+, CD69+, and CD127+, individually, and about 15% or less (e.g., less than 15%, less than 10%, less than 9%, less than 8%, less than 7%, less than 6%, less than 5%, less than 4%, less than 3%, less than 2%, less than 1%) of the isolated somatic stem cell population express CD45, CD3, CD19, CD34, CD44, TIM3, LAG3, and CTLA4, individually.

In some embodiments, at least about 50% to 99% (e.g., at least 50%, at least 60%, at least 65%, at least 70%, at least 75%, at least 80%, at least 85%, at least 90%, at least 95%, or at least 99%, about 55%, about 60%, about 70%, about 75%, about 80%, about 85%, about 90%, about 91%, about 92%, about 93%, about 94%, about 95%, about 96%, about 97%, about 98%, or about 99%) of the cells within the isolated somatic stem cell population are about 6.0 μm or less in size, e.g., about 0.5 μm to 6.0 μm , about 2.0 μm to less than 6.0 μm , or about 1.5 to 5.5 μm. In some embodiments, after a period of culture in a non-differentiating cell medium, the sizes of the cells in the stem cell population may increase to ranging from about 6.0 μm to about 25 μm in diameter. Thus, the isolated somatic stem cell population of this disclosure also encompasses a population including cells larger than 6.0 μm in size (e.g., about 6.0 μm to about 25 μm ) after being cultured in a non-differentiating medium but substantially maintains the expression profile of the population before culturing.

In one embodiment, the isolated somatic stem cell population of the disclosure substantially maintains its properties with respect to the positive or negative expression of a marker of interest (e.g., PD-L1, Lgr5, CD45, CD3, CD19, CD34, CD61, HLA-ABC, CD44, CD69, CD127, TIM3, LAG3, or CTLA4) after cultivation to at least 4^{th} passage (e.g., 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, or further passage) and substantially maintains the cell marker expression after the at least 4^{th} passage (e.g., 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, or further passage).

In one embodiment, the isolated somatic stem cell population of the disclosure remains substantially identical in expression profile after cryopreservation and thawing.

In another aspect, the disclosure provides a composition comprising the isolated somatic stem cell population as disclosed herein. The composition may further contain one or more pharmaceutically acceptable carriers. Examples of carriers include preservative, solubilizer, and stabilizer. The composition may be used for non-oral administration, for example intravenous, subcutaneous, or intra-peritoneal administration or topical application. A dosage of the cell population may vary in accordance with different factors, such as type of disease, degree of seriousness of disease, administration route, or weight, age and sex of subject.

For example, a single unit dose of the isolated somatic stem cells can comprise, in various embodiments, about, at least, no more than, or more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ stem cells. In some embodiments, at least 50% (e.g., about 50% to at least 90%) of the cells are viable or living.

The pharmaceutical compositions of the disclosure can comprise one or more compounds that, e.g., facilitate engraftment (e.g., anti-T-cell receptor antibodies, an immunosuppressant, or the like); stabilizers such as albumin, dextran 40, gelatin, hydroxyethyl starch, and the like.

### Preparation of an Isolated Somatic Stem Cell Population

The isolated somatic stem cell population in this disclosure is prepared from non-embryonic origins. These cells can be isolated either from a human or from a non-human subject. Examples of a non-human source include, but are not limited to non-human primates, dogs, rodents, guinea pigs, cats, horses, cows, sheep, and pigs. In one embodiment, the isolated somatic stem cell population is derived from a human.

The isolated somatic stem cell population can be prepared from a tissue sample. The tissue sample can be a bodily fluid sample derived from a bodily fluid (e.g., peripheral blood sample, whole blood sample, or bone marrow sample) obtained from a subject. In some embodiments, a bodily fluid sample drawn from a subject can be used as is as a tissue sample for preparing the somatic stem cell population. Optionally, the bodily fluid sample may be first incubated with a red-blood-cell (RBC) lysis buffer for a time period under suitable conditions and subjected to centrifugation to remove cell debris to prepare a tissue sample for isolating the stem cells.

In some embodiments, the tissue sample can be derived from a solid tissue sample (e.g., muscle sample or adipocyte sample) obtained from a subject. If the tissue sample is obtained from a solid tissue, collagenase may be added to treat the solid tissue sample for at least 6 or 8 hours at a suitable temperature (e.g., about 37° C., greater than 30° C., between 35° C. and 39° C., or between 30° C. and 40° C.). The treated solid tissue sample can then be then subjected to centrifugation at a low speed (e.g., 100 g, between 50 g and 450 g, or between 80 g and 250 g) for a time period (e.g., about 10 to 20 minutes) and then at a high speed (e.g., 1,000 g, greater than 700 g, greater than 1,300 g, between 500 g and 2,500 g, or between 800 g and 1,600 g) for a time period (e.g., about 10 minutes and 30 minutes) so as to obtain a sample containing small cells. The sample containing small cells can be suspended in a suitable medium (e.g., PBS and BSA) to obtain a tissue sample.

An isolated somatic stem cell population can be prepared from a tissue sample using the following procedure. First, the tissue sample is incubated with a divalent cation chelating agent (e.g., EDTA, EGTA, and sodium citrate) or heparin in a container until the sample is separated into an upper layer and a lower layer. In some embodiments, the tissue sample can be placed in an anti-clotting EDTA tube or heparin tube. The incubation can be performed for 6 to 72 hours at about 4° C. to 12° C. For example, the incubation can be performed for 12, 24, 30, 36, 42, 48, 54, 60, 66, or 72 hours at 4° C.

The upper layer produced by the above incubating step is collected, which includes a population of somatic stem cells that are about 6 micrometers or less in size. This population of somatic stem cells can be referred to as SB cells, which includes subpopulations of somatic stem cells characterized by particular marker expression profiles.

The collected upper layer can be subjected to additional steps to further isolate a desired population of somatic stem cells using methods known in the art based on cell size (e.g., centrifuging and filtering) or based on cell surface markers (e.g., flow cytometry). For example, cells in the size range of about 1.5 or 2.0 μm to about 6.0 μm can be positively selected and cells/particles less than about 1.5 to 2.0 μm can be negatively selected to remove microparticles or microvesicles.

In some embodiments, the incubating step can be performed by incubating the tissue sample with heparin, and in between the collecting step and the isolating step, the collected upper layer is further incubated with EDTA.

Optionally, the preparation method can further include, after the upper layer has been collected, incubating it with ADP to allow platelets and/or microparticles to precipitate for further enrichment of somatic stem cells.

It was observed that the percentage of cells having sizes between about 1 μm to 6.0 μm in the upper layer prepared using an EDTA tube was significantly higher (e.g., more than 20 folds) than that prepared using a heparin tube. The results suggest that EDTA increases the number of small somatic stem cells. If the collected upper layer prepared in a heparin tube was further incubated in an EDTA tube, an increase in the number of small cells was also observed. The possibility that the increase in cell number in the EDTA tube was due to an increase in the number of platelets and microparticles was tested and ruled out. While not intended to be bound by theory, it is possible that EDTA can chelate other divalent ions and activate stem cells that are in G0 phase and force the stem cells to proliferate and expand.

Enrichment of somatic stem cells positively or negatively expressing a marker of interest (e.g., PD-L1 or Lgr5) can be carried out by subjecting the isolated population prepared from the upper layer to positive or negative selection by, for example, fluorescence activated cell sorting (FACS).

The isolated population of somatic stem cells can be further propagated in a non-differentiating medium culture for more than 10, 20, 50, 100 or more population doublings without indications of spontaneous differentiation, senescence, morphological changes, increased growth rate, or changes in ability to differentiate. These stem cells can be stored by standard methods before use.

### Uses of the Isolated Somatic Stem Cell Population

In another aspect, the disclosure provides a method for modulating an immune response in vitro or in vivo, comprising contacting the isolated somatic stem cell population with immune cells (e.g., T cells), or administering an effective amount of the isolated somatic stem cell population of this disclosure to a subject in need thereof.

In some embodiments of the methods of this disclosure, the isolated somatic stem cell population is allogeneic or autologous.

In yet other embodiments of the disclosure, the method is for inhibiting an immune response of T cells. For example, activation of T cells and other functions of T cells (e.g., cytotoxicity, release of cytokines, or proliferation) can be suppressed by contacting the T cells with the isolated somatic stem cell population of the disclosure. In some further embodiments of the disclosure, the method is for reducing the number of T cells. In some embodiments of the disclosure, the method is for increasing PD-1 expression in the T cells.

In some embodiments of the disclosure, the T cells express CD25, CD69, CD3, CD4, CD8, CD45RA, CD45RO, PD-1, CTLA4 and/or Foxp3 prior to being contacted with the isolated somatic stem cell population. In some embodiments of the disclosure, the T cells are naïve or activated. In some embodiments, after being contacted with the isolated somatic stem cell population, expression of one or more markers for activated T cells, such as CD45, CD25, and CD69, is reduced as compared to before the contacting step.

Prior to administering the isolated population of somatic stem cells to a subject, a sample (e.g., a blood sample) obtained from the subject can be assayed to detect, quantify, or characterize the T cells or immune responses in the subject using methods known in the art. The results can be used to determine whether the subject is in need of or suitable for treatment with the isolated somatic stem cell population for immunosuppression. For example, a subject showing higher than normal T cell activities or overactive immune responses may be in need of or is suitable for the treatment.

CD25 (α chain of the high-affinity IL-2 receptor) is a protein that in humans is encoded by the IL2RA gene. The interleukin 2 (IL2) receptor alpha (IL2RA) and beta (IL2RB) chains, together with the common gamma chain (IL2RG), constitute the high-affinity IL2 receptor. Homodimeric alpha chains (IL2RA) result in low-affinity receptor, while homodimeric beta (IL2RB) chains produce a medium-affinity receptor.

CD69 is a membrane-bound, type II C-lectin receptor. It is a classical early marker of lymphocyte activation due to its rapid appearance on the surface of the plasma membrane after stimulation. CD69 is expressed by several subsets of tissue resident immune cells, including resident memory T (TRM) cells and gamma delta (γδ) T cells, and is therefore considered a marker of tissue retention.

CD3 is a protein complex and T cell co-receptor that is involved in activating both the cytotoxic T cell (CD8⁺naive T cells) and T helper cells (CD4⁺ naive T cells).

CD4 is a membrane glycoprotein of T cells. The CD4 antigen acts as a coreceptor with the T-cell receptor on the T cells to recognize antigens displayed by an antigen presenting cell in the context of class II MHC molecules.

CD8 is a cell surface glycoprotein found on most cytotoxic T cells that mediates efficient cell-cell interactions within the immune system. CD8 acts as a coreceptor with the T-cell receptor on the T cells to recognize antigens displayed by an antigen presenting cell in the context of class I MHC molecules.

CD45, also known as protein tyrosine phosphatase, receptor type, C also known as PTPRC is an enzyme that, in humans, is encoded by the PTPRC gene. CD45RA is located on naive T cells and CD45RO is located on memory T cells. CD45 is also highly glycosylated. CD45RA is located on naive T cells. Naive T lymphocytes express large CD45 isoforms and are usually positive for CD45RA. CD45RO facilitates T cell activation.

As used herein, programmed cell death protein-1, also known as PD-1and CD279, is a cell surface protein and an immune checkpoint. It can regulate the immune system and promote autoimmune function, and inhibit tolerance produced by inflammatory activity of T cells. While not wishing to be limited by theory, it is believed that a receptor of PD-1 can be expressed on the surface of activated T cells and B cells, and therefore the interaction between the PD-L1 protein expressed on the isolated somatic stem cells of this disclosure and the PD-1 receptor provides inhibitory signals of regulating cell activation and proliferation. In addition, the isolated somatic stem cells can potentially inhibit the proliferation and function of T cells through direct contact with activated and naive T cells and indirect secretion of soluble PD-L1.

CTLA4 (cytotoxic T-lymphocyte-associated protein 4), also known as CD152 (cluster of differentiation 152), is a protein receptor that functions as an immune checkpoint and downregulates immune responses.

Foxp3 (forkhead box P3), also known as scurfin, is a protein involved in immune system responses. FOXP3 appears to function as a master regulator of the regulatory pathway in the development and function of regulatory T cells.

In some embodiments of the disclosure, in the method for modulating an immune response of T cells, the ratio of the cell count of the T cells to the cell count of the isolated population of somatic stem cells can range from 10:1 to 1:10, from 5:1 to 1:10, from 1:1 to 1:10; from 1:2 to 1:9, from 1:3 to 1:8, from 1:4 to 1:7, or from 1:5 to 1:6.

In a further aspect, the disclosure provides a method for inhibiting or reducing the risk of an alloresponse or autoimmune response in a subject in need thereof, comprising administrating an effective amount of the isolated somatic stem cell population as disclosed herein to the subject.

In some embodiments of the methods described herein, a subject being administered the isolated somatic stem cell population of this disclosure has a condition or is at risk of developing a condition (e.g., an autoimmune disorder, inflammatory disorder, or transplant rejection) that can benefit from immunosuppressive therapy. In some embodiments, the condition is a T-cell mediated autoimmune disease.

The isolated somatic stem cell population of this disclosure can be used to treat autoimmune or inflammatory condition or disease, including but not limited to, multiple sclerosis (MS), type 1 diabetes, myocarditis, graft-versus-host disease, rheumatoid arthritis (RA), celiac disease, psoriasis, alopecia areata, lupus (e.g., systemic lupus erythematosus (SLE)), Sjögren's syndrome, psoriasis, psoriatic arthritis, transplant rejection, dermatomyositis, scleroderma, vasculitis, rheumatoid vasculitis, urticarial vasculitis, vitiligo, inflammatory bowel disease, Crohn's disease, ulcerative colitis, Addison's disease, and Graves'disease.

The condition, disease or disorder, for example, can be graft-versus-host disease. The graft-versus-host disease may develop after an allogeneic bone marrow transplant, a solid organ transplant, or a composite tissue allograft. In some embodiments, said graft-versus-host disease in the subject is reduced in grade by at least one step by administration of the isolated somatic stem cell population.

In another embodiment, said disease, disorder or condition is rheumatoid arthritis (RA). Administration of the isolated somatic stem cell population of the disclosure to a subject may be sufficient to cause a detectable improvement in one or more symptoms of RA, or sufficient to detectably reduce the onset of one or more symptoms of RA, in at least one joint in the individual with RA. The administration can be sufficient to cause a detectable improvement in one or more symptoms of RA, or sufficient to detectably reduce the onset of one or more symptoms of RA, in at least one non-joint tissue in the individual with RA. The non-joint tissue can be skin (dermis), lungs, autoimmune system or blood, renal tissue, cardiovascular tissue, ocular tissue, or neurological tissue. In some embodiments, said symptom of RA is a condition adjunct to RA, e.g., pyoderma gangrenosum, neutrophilic dermatosis, Sweet's syndrome, viral infection, erythema nodosum, lobular panniculitis, atrophy of digital skin, palmar erythema, diffuse thinning (rice paper skin), skin fragility, subcutaneous nodules on an exterior surface, e.g., on the elbows, fibrosis of the lungs (e.g., as a consequence of methotrexate therapy), Caplan's nodules, vascular disorders, nail fold infarcts, neuropathy, nephropathy, amyloidosis, muscular pseudohypertrophy, endocarditis, left ventricular failure, vasculitis, scleromalacia, mononeuritis multiplex, atlanto-axial subluxation.

In another specific embodiment, the disease, disorder or condition is multiple sclerosis. In a more specific embodiment, said multiple sclerosis is relapsing/remitting multiple sclerosis, secondary progressive multiple sclerosis, primary progressive multiple sclerosis, or progressive/relapsing multiple sclerosis.

In another specific embodiment, the disease, disorder or condition is lupus erythematosus. In a more specific embodiment, said symptom of lupus erythematosus is one or more of malar rash, butterfly rash, discoid lupus, alopecia, mouth, nasal, and vaginal ulcers, lesions on the skin, joint pain anemia and/or iron deficiency, lower than normal platelet and white blood cell counts, antiphospholipid antibody syndrome, presence of anticardiolipin antibody in the blood, pericarditis, myocarditis, endocarditis, lung and/or pleural inflammation, pleuritis, pleural effusion, lupus pneumonitis, chronic diffuse interstitial lung disease, pulmonary hypertension, pulmonary emboli, pulmonary hemorrhage, painless hematuria or proteinuria, lupus nephritis, renal failure, and/or development of membranous glomerulonephritis with “wire loop” abnormalities); neurological manifestations (e.g., seizures, psychosis, abnormalities in the cerebrospinal fluid); T-cell abnormalities (e.g., deficiency in CD45 phosphatase and/or increased expression of CD40 ligand); and/or nonspecific manifestations (e.g., lupus gastroenteritis, lupus pancreatitis, lupus cystitis, autoimmune inner ear disease, parasympathetic dysfunction, retinal vasculitis, systemic vasculitis, increased expression of Fc.epsilon.RI.gamma., increased and sustained calcium levels in T cells, increase of inositol triphosphate in the blood, reduction in protein kinase C phosphorylation, reduction in Ras-MAP kinase signaling, and/or a deficiency in protein kinase A I activity.

In another specific embodiment, said disease, disorder or condition is scleroderma. In a more specific embodiment, the scleroderma is diffuse scleroderma. In another specific embodiment, said disease, disorder or condition is mycosis fungoides (Alibert-Bazin syndrome). In another embodiment, said disease, disorder or condition is diabetes. In another embodiment, said disease, disorder or condition is psoriasis. In a more specific embodiment, the psoriasis is plaque psoriasis (psoriasis vulgaris).

In another specific embodiment of any of the above methods, the method comprises administration of a second therapeutic agent to the individual having the disease, disorder or condition. In a more specific embodiment, said second therapeutic agent is an anti-inflammatory agent, an immunomodulatory agent, and immunosuppressive agent, a pain medication, or an antibiotic. In one embodiment, the isolated somatic stem cell population is contacted with the T cells in vivo in an individual, and a composition comprising an immunosuppressive agent is administered to the individual. Immunosuppressive agents are well-known in the art and include, e.g., anti-T cell receptor antibodies (monoclonal or polyclonal, or antibody fragments or derivatives thereof), anti-IL-2 receptor antibodies (e.g., Basiliximab (SIMULECT®) or daclizumab (ZENAPAX)®), anti T cell receptor antibodies (e.g., Muromonab-CD3), azathioprine, corticosteroids, cyclosporine, tacrolimus, mycophenolate mofetil, sirolimus, calcineurin inhibitors, and the like. In a specific embodiment, the immunosuppressive agent is a neutralizing antibody to macrophage inflammatory protein (MIP)-1α or MIP-1β. The anti-MIP-1α or MIP-1β antibody can be administered in an amount sufficient to cause a detectable reduction in the amount of MIP-1α and/or MIP-1β in said individual, e.g., at the time of transplanting.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All patents, patent applications, and other publications (e.g., scientific articles, books, websites, and databases) mentioned herein are incorporated by reference in their entirety.

### Example 1

### Isolation of a Population of Small Somatic Stem Cells

A blood sample or a bone marrow sample was drawn from a person and placed in an anti-clotting EDTA tube or heparin tube. After incubating the tube for 6 to 48 hours at 4° C., the sample separated into two layers. The top layer contained a somatic stem cell population, which was further analyzed by methods such as C6 accuri flow cytometry, immunocytochemistry, and RNA extraction/RT-PCR. The bottom layer contained red and white blood cells, which are not smaller than 6.0 μm .

Particles in the top layer were analyzed by the size beads of flow cytometry. It was found that they were no bigger than 6.0 μm . It is known that platelets and microparticles are smaller than 6.0 μm but have no nuclei and therefore cannot be stained by DAPI or SYTO. To examine the particles, DAPI and SYTO staining were carried out. The results showed that many particles were stained positive by both dyes, i.e., DAPI and SYTO, suggesting that these particles were cells containing DNA nuclei, but not platelets and microparticles. It was further found that the DAPI-negative particles were about 0.01 to 1.5 μm . These results suggest that the top layer contained stem cells, platelets, and microparticles. It was confirmed that the DAPI-positive particles were indeed cells containing an integral chromosome structure.

It was also confirmed that the population of small somatic stem cells is capable of differentiating into ectoderm (e.g., neural cells and epidermal cells), mesoderm (e.g., adipocytes, osteogenic cells, and muscle cells), and endoderm cells (e.g., hepatocyte and islet cells).

### Example 2

### Characterization of an Isolated Population of Small Somatic Stem Cells

Exemplary isolated somatic stem cell populations were prepared from peripheral blood samples collected from six individuals using the procedure described in Example 1.

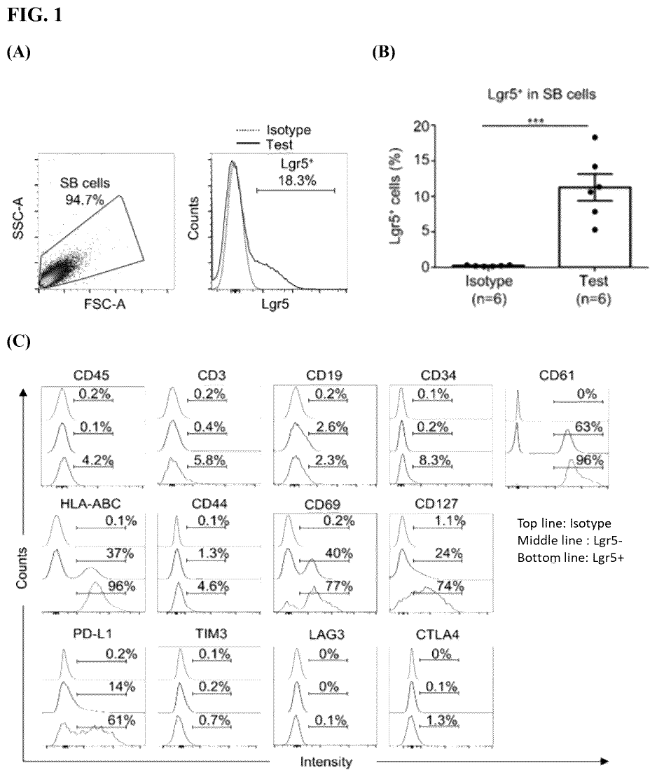

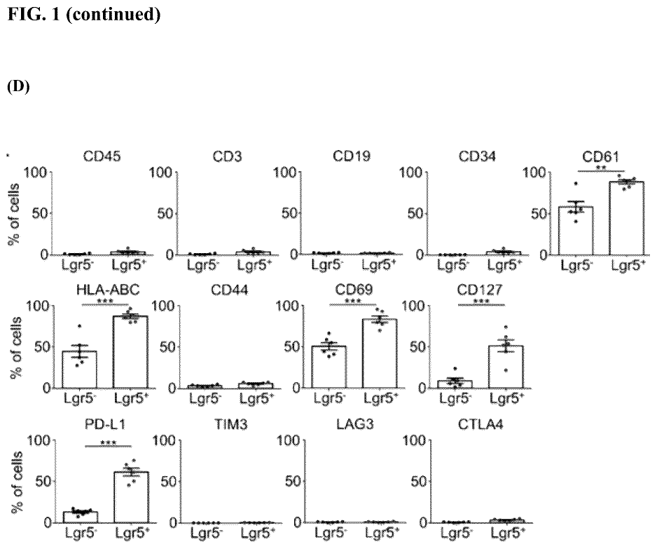

The isolated population of somatic stem cells (SB cells) was subjected to fluorescence-activated cell sorting (FACS) analysis. Lgr5, a stem cell marker in the intestine, was found to be expressed in the cell population. It was estimated that approximately 6%-18% (depending on the individual) of the cells expressed the Lgr5 marker. See FIG. 1, (A) and (B). Next, whether expression of Lgr5 would affect the cells' characteristics was investigated. Expression of a stemness marker (CD34), several hematological cell markers (CD45, CD3, CD19, CD34, and CD61) and immune-related markers (HLA-ABC, CD44, CD69, CD127, PD-L1, TIM3, LAG3, and CTLA4) were determined. It was found that Lgr5+ cells highly expressed CD61, HLA-ABC, CD69, CD127, and PD-L1, compared to Lgr5-cells. See FIG. 1, (C) and (D). In addition, among both the Lgr5+ and Lgr5-cells, there was low or no expression of each of CD45, CD3, CD19, CD34, CD44, TIM3, LAG3, and CTLA4. See FIG. 1, (C) and (D). The lack of the expression of CD45, a cell surface glycoprotein present on all nucleated hematopoietic cells, suggests that these somatic stem cells may not belong to the nucleated hematopoietic cell lineage, such as leukocytes. However, the expression of CD69 and CD127 suggests that these cells may be associated with T cells.

Intriguingly, PD-L1, which plays a key role in suppressing the immune response by binding to the PD-1 receptor on T cells, was found to be highly expressed in the isolated SB cell population, particularly in the Lgr5+ cell population. This finding indicates that the population of somatic stem cells may have immunoregulatory functions, particularly concerning T cells.

### Example 3

### Immunogenicity and Immunomodulating Properties of the Isolated Population of Somatic Stem Cells

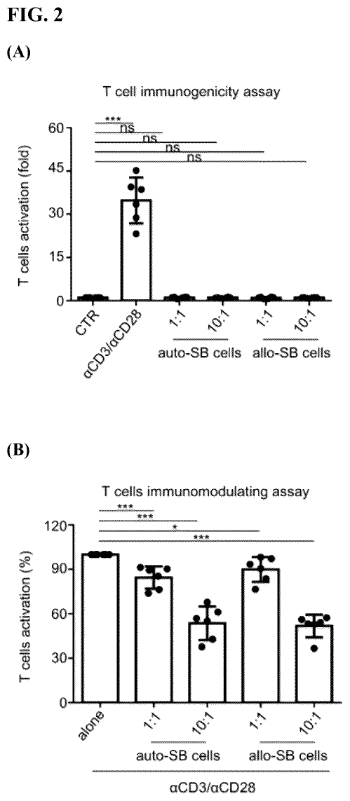

Immunogenicity is one of the critical considerations in allogeneic cell transplantation. Therefore, an analysis of immunogenicity was conducted by co-culturing the isolated population of somatic stem cells with allogeneic human T cells in vitro. Notably, after co-culturing T cells with allogeneic somatic stem cells, no significant proliferation in T cells was observed in the 1:1 or 1:10 ratios (T cells to somatic stem cells), indicating that these somatic stem cells possess low immunogenicity characteristics despite being confirmed to highly express HLA-ABC. See FIG. 2 (A). In addition, as it was found that the isolated population of somatic stem cells express PD-L1, a ligand for immune checkpoints on T cells, whether the somatic stem cells would affect T cell activity was assessed. It was observed that the stem cells significantly inhibited T cell activation, which was induced by anti-CD3 and anti-CD28 antibodies. See FIG. 2(B). This effect was observed even with allogeneic somatic stem cells. See FIG. 2(B).

The results showed that the isolated population of somatic stem cells exhibit low immunogenicity characteristics, suggesting the potential application of allogeneic stem cells in cell therapy or transplantation. Additionally, the stem cell population has the ability to inhibit T cell activity, which suggests that the isolated stem cell population could be used in cell therapy for certain T-cell-mediated immune diseases.

### Example 4

### Induction of PD-1 Positive Population in Activated T Cells Upon Co-Culturing With Isolated Population of Somatic Stem Cells

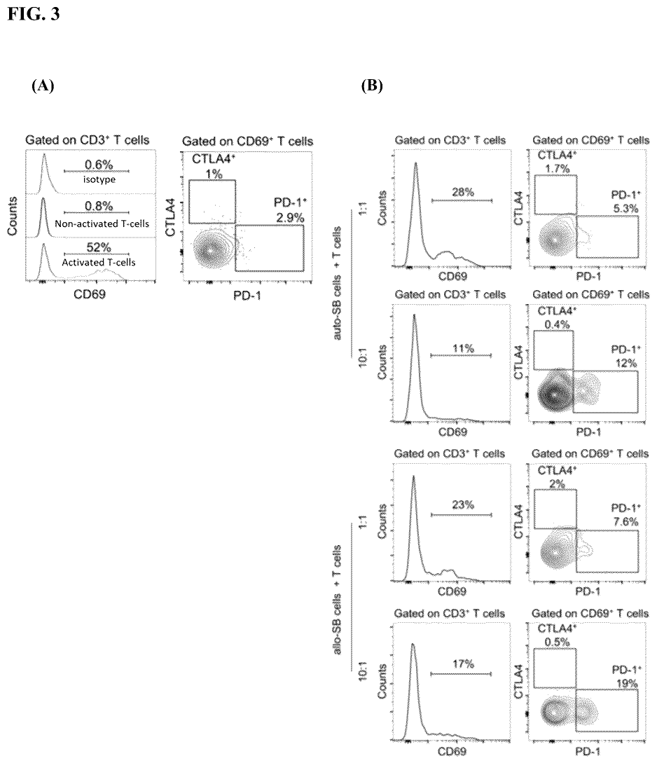

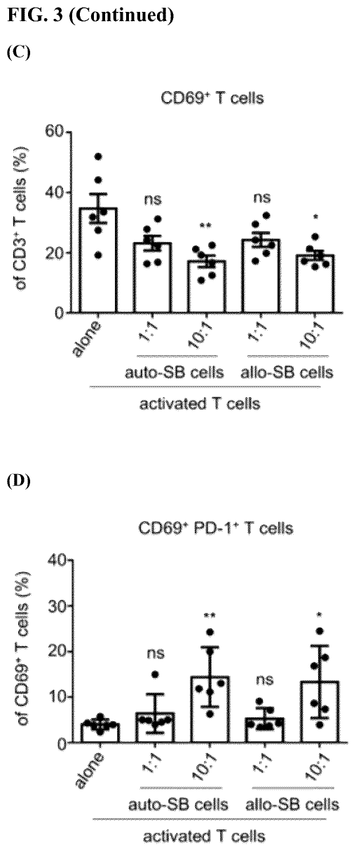

To further investigate the effects of the isolated population of somatic stem cells on T cells, test was done to investigate the T cell activation marker (CD69) and immune checkpoint markers (CTLA4 and PD-1) on CD3+ T cells after co-culturing with the stem cell population under activation conditions using anti-CD3 and anti-CD28 antibodies (αCD3/αCD28 activation). Induction of the CD69+ population was significantly increased after αCD3/αCD28 activation for 3 days, with low or no expression of CTLA4 and PD-1 in CD69+ T cells. See FIG. 3. Importantly, after co-culturing with the stem cell population, there was a significant decrease in the CD69+ T cell population. A notable increase in the PD-1+ population within CD69+ T cells (PD-1+/CD69+ T cells) was simultaneously observed after 3 days of activation. See FIG. 3. These data suggest that the isolated somatic stem cell population could influence T cell activity by inhibiting both the activation and proliferation of T cells, possibly through the engagement of PD-1 and PD-L1 immune checkpoint signals.

### Example 5

### Immunosuppressive Activity of PD-L1+ Somatic Stem Cells

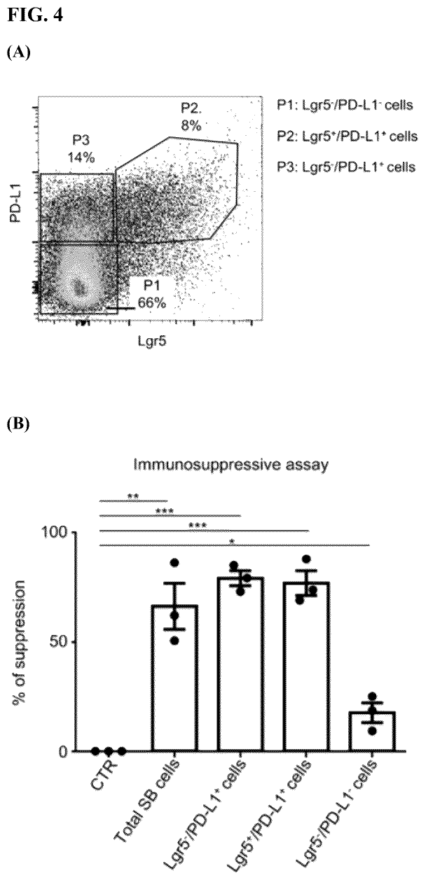

To further verify the role of PD-L1 in inhibiting T cell activation by the stem cells, cell sorting of the isolated somatic stem cell population was performed based on the expression of Lgr5 and PD-L1, including Lgr5−/PD-L1− cells (Population 1, P1), Lgr5+/PD-L1+ cells (Population 2, P2), and Lgr5−/PD-L1+ cells (Population 3, P3). See FIG. 4(A). The three populations were individually co-cultured with T cells for 3 days under αCD3/αCD28 activation. The results showed that both Lgr5+/PD-L1+ and Lgr5−/PD-L1+ stem cells exhibited significant suppressive activity on T cells; however, total SB cells and Lgr5−/PD-L1-stem cells showed much lower suppressive effects in comparison. See FIG. 4(B). Although there were too few Lgr 5+/PD-L1− cells (approximately 2-3% of the isolated stem cell population) to obtain sufficient cells for co-culture experiments effectively, the data still indicate that the immunosuppressive function of the majority of PD-L1− negative stem cells is significantly lower than that of PD-L1− positive stem cells.

The results demonstrate that the suppressive effect of the isolated stem cell population on T cells largely depends on the expression of PD-L1. This finding suggests cell selection strategies on the isolated stem cell population when the intended application is for treatment of immune disorders, in particular T-cell-mediated inflammatory diseases.

What is claimed is:

1. An isolated somatic stem cell population, comprising somatic stem cells that are about 6 μm or less in size and are PD-L1+.

2. The isolated somatic stem cell population of claim 1, wherein at least about 50% of the cells in the somatic stem cell population are PD-L1+.

3. The isolated somatic stem cell population of claim 2, further comprising Lgr5+ cells.

4. The isolated somatic stem cell population of claim 3, wherein at least about 50% of the Lgr5+ cells are PD-L1+.

5. The isolated somatic stem cell population of claim 4, wherein at least about 80% of the cells in the somatic stem cell population are Lgr5+.

6. The isolated somatic stem cell population of claim 3, wherein at least about 80% of the cells in the somatic stem cell population are PD-L1+.

7. The isolated somatic stem cell population of claim 3, wherein at least about 90% of the cells in the somatic stem cell population are PD-L1+.

8. The isolated somatic stem cell population of claim 3, further comprising somatic stem cells that are CD61+, HLA-ABC+, CD69+, and/or CD127+.

9. The isolated somatic stem cell population of claim 1, wherein the isolated somatic stem cell population is prepared using a process including:
incubating a peripheral blood or bone marrow sample with EDTA or heparin in a container until the sample is separated into an upper layer and a lower layer;
collecting the upper layer, and
isolating from the upper layer a population of somatic stem cells containing somatic stem cells that are about 6.0 μm or less in size and PD-L1+.

10. The isolated somatic stem cell population of claim 9, wherein the isolating step includes positive selection for PD-L1+ cells.

11. A composition comprising the isolated somatic stem cell population of claim 1.

12. A method of preparing the population of somatic stem cells of claim 1, the method comprising:
incubating a tissue sample with EDTA or heparin in a container until the sample is separated into an upper layer and a lower layer;
collecting the upper layer, and
isolating from the upper layer a population of somatic stem cells containing somatic stem cells that are about 6.0 μm or less in size and PD-L1+.

13. A method of modulating an immune response, comprising contacting immune cells with an effective amount of the isolated somatic stem cell population of claim 1.

14. The method of claim 13, wherein the immune cells are T cells and the immune response is inhibited.

15. The method of claim 14, wherein the contacting step is carried out in vitro.

16. The method of claim 14, wherein the contacting step includes administering an effective amount of the isolated somatic stem cell population to a subject in need thereof.

17. The method of claim 16, wherein the subject has or is at risk of developing an alloresponse or autoimmune response.

18. The method of claim 16, wherein the subject has or is at risk of developing an autoimmune disorder; optionally, the autoimmune disorder is a T-cell-mediated autoimmune disorder.

19. The method of claim 13, wherein the isolated somatic stem cell population is allogeneic or autologous.
